(19) United States
(12) Patent Application Publication
(10) Pub. No.: US 2025/0161564 A1
(43) Pub. Date: May 22, 2025

(54) PREVENTING OVERINFUSION IN PROGRAMMABLE INFUSIONS

(71) Applicant: B. Braun Medical Inc., Bethlehem, PA (US)

(72) Inventors: William Contreras, Nutley, NJ (US); Michael Zakrewski, Carrollton, TX (US)

(73) Assignee: B. Braun Medical Inc., Bethlehem, PA (US)

(21) Appl. No.: 18/516,344

(22) Filed: Nov. 21, 2023

(51) Int. Cl. A61M 5/168

(52) U.S. Cl. CPC A61M 5/1684; A61M 2205/3334; A61M 2205/3379

(57) ABSTRACT

To prevent overinfusion, an infusion pump restricts the volume of infusate that the patient can receive over a given period of time by utilizing an analytical model of infusate volume delivered to the patient as a function of time that takes the form of a piecewise function, v(t). During initial programming of an infusion, an initial version of the model is created. The model is updated each time the flow rate modulation pattern is modified over the course of the infusion. Each update to the model results in one or more new elements being added to the piecewise function. The maxima of each element of the piecewise Δv(t) is determined and compared to a maximum permissible volume set by the user. If any of the maxima meet or exceed the value, an alert is issued. Once the maximum permissible volume is reached, the infusion is stopped.

### FIELD OF THE INVENTION

The present disclosure is related to infusion pumps and, more particularly, to methods for preventing overinfusion during programmable infusions by infusion pumps.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids is desirable to optimize treatment of a patient as there are many fluids where small variations can be critical.

Infusion pumps are typically set up and programmed by medical professionals in a medical setting. However, ambulatory infusion pumps may be operated by a patient in a home setting. Safeguards to prevent overinfusion are desirable for safe programming and operation of infusion pumps, particularly when the infusion pumps are operated by patients without the assistance of medical professionals.

### SUMMARY

Examples described herein are directed to methods implemented by infusion pumps for preventing overinfusion of fluids. In sample configurations, an infusion pump and corresponding methods and computer-readable media are provided that are implemented on an infusion pump including a pump motor configured to pump a liquid through a tube, a power source for the pump motor, and at least one pump controller for controlling the pump motor. The at least one pump controller includes a processor and a memory storing instructions therein that, when executed by the at least one pump controller, perform operations including enabling input of a flow rate modulation pattern including a maximum permissible infusate volume and creating a model including a piecewise function Δv(t) having a value for any given time t that is equal to a volume of fluid delivered by the pump motor to a user over a past time T. The model is designed to account for the flow rate modulation pattern including any infusion components (such as loading doses and boluses) specified in the flow rate modulation pattern. A maxima of each element of the piecewise function Δv(t) is determined, and the maxima of each element of the piecewise function Δv(t) is compared to the maximum permissible volume. When any of the maxima meet or exceed the maximum permissible volume, an alert is issued to alert the user to a possible overinfusion. If the user wishes to continue the infusion, the fluid is provided to the user in accordance with the flow rate modulation pattern until the maximum permissible volume is reached. The at least one pump controller may execute additional instructions to perform additional operations including monitoring the volume of fluid delivered by the pump motor over the past time T over a course of an infusion, and when the maximum permissible volume is reached or the user chooses not to proceed, stopping the infusion.

The at least one pump controller may further execute instructions to perform additional operations including enabling the flow rate modulation pattern to be updated over the course of an infusion and updating the model each time the flow rate modulation pattern is modified by adding one or more new elements to the piecewise function Δv(t). The model may include the piecewise function Δv(t)=v(t)−v(t−T). On the other hand, to save on memory usage, the model may include the piecewise function Δv(t)=∫₀^{t}u(t−τ)ƒ(τ)dτ, where u(t−τ) is a Boxcar function with a width equal to T and a height equal to 1 and ƒ(τ) is a function representative of the flow rate modulation pattern.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a diagram illustrating a sample flow rate modulation pattern specified during initial programming of an infusion, comprising a loading dose followed by a basal rate.

FIG. 2 is a diagram illustrating a plot of infusate volume delivered to the patient as a function of time, corresponding to the flow rate modulation pattern of FIG. 1.

FIG. 3 illustrates the flow rate modulation pattern of FIG. 1, modified to account for a bolus requested at time t_{CS}.

FIG. 4 is a plot of infusate volume delivered to the patient as a function of time corresponding to the flow rate modulation pattern of FIG. 3.

FIG. 5 is a perspective view of an example ambulatory peristaltic infusion pump.

FIG. 6 is a perspective view of an example cassette with a free flow prevention clamp for use with the ambulatory peristaltic infusion pump of FIG. 5.

FIG. 7 is a partial perspective view of the ambulatory peristaltic infusion pump of FIG. 5 illustrating cams that engage the free flow prevention clamp when the cassette is coupled to the ambulatory peristaltic infusion pump.

FIGS. 8 and 9 are cutaway views of the ambulatory peristaltic infusion pump of FIG. 5 illustrating pump sliders and cam rods for moving the pump sliders.

FIG. 10 is a diagram illustrating the communication module and multiple controller cores that together ensure proper operation of the ambulatory peristaltic infusion pump in a sample configuration.

FIG. 11 is a flow chart illustrating the combined operation of the communication application of the communication module and software running on the user interface controller core for safe operation of the ambulatory peristaltic infusion pump in a sample configuration.

FIG. 12 is a diagram illustrating Δv(t) for the flow rate modulation pattern of FIG. 1.

FIG. 13 is a flow chart illustrating a method for preventing overinfusion by the infusion pump in a sample configuration.

FIG. 14 is a functional block diagram illustrating a general-purpose computer hardware platform configured to implement the functional examples described with respect to FIGS. 1-13.

FIG. 15 is another functional block diagram illustrating a general-purpose computer hardware platform configured to implement the functional examples described with respect to FIGS. 1-13.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the overinfusion safeguards described herein may be used with a variety of other pump types.

Those skilled in the art will appreciate that an infusion can be treated as a process wherein a container (or sequence of containers) of infusate is drained (in a controlled fashion) into a patient. The rate at which the infusate is drained is modulated to achieve a particular therapeutic effect.

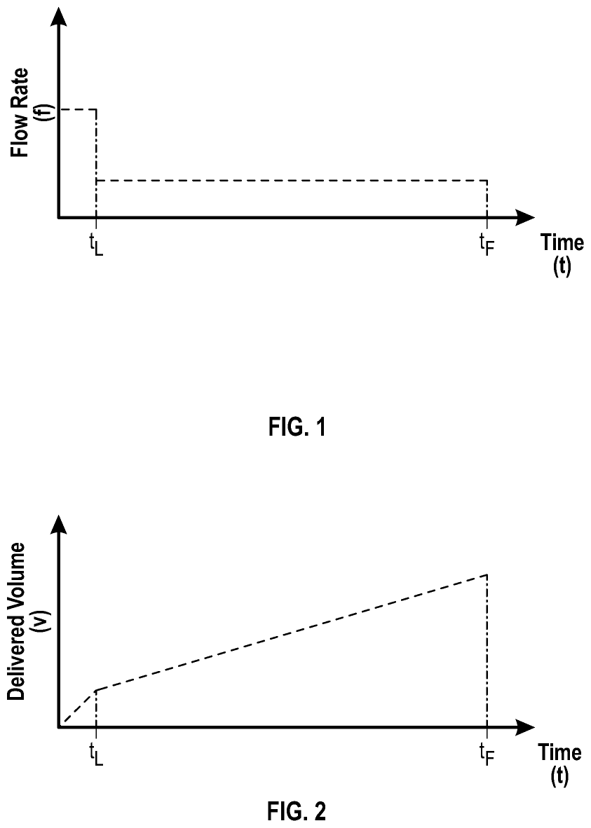

In cases where an electronic pump is used to administer an infusion, the rate at which infusate is drained from the container can be modulated via the pump user interface. At the outset of such an infusion, an initial modulation pattern is specified for the infusion. The user may specify, for instance, that a loading dose (during which infusate is administered at a high rate to bring the concentration of infusate in the patient's bloodstream up quickly) be performed, followed by a basal rate (which is a reduced rate to achieve a steady therapeutic effect). A plot of such a modulation pattern is shown in FIG. 1, which illustrates the sample flow rate modulation pattern specified during initial programming of an infusion, comprising a loading dose through time t_{L} followed by a basal rate through time t_{F}. It should be noted that, in FIG. 1, flow rate is shown as a dotted line to indicate that the values are projected and that the infusion has not actually run yet. It should also be noted that FIG. 1 is from the perspective of the patient (to which infusate is being delivered) and not the container (from which infusate is being drained), which is why flow rate is positive. A corresponding plot of infusate volume delivered to the patient as a function of time is shown in FIG. 2.

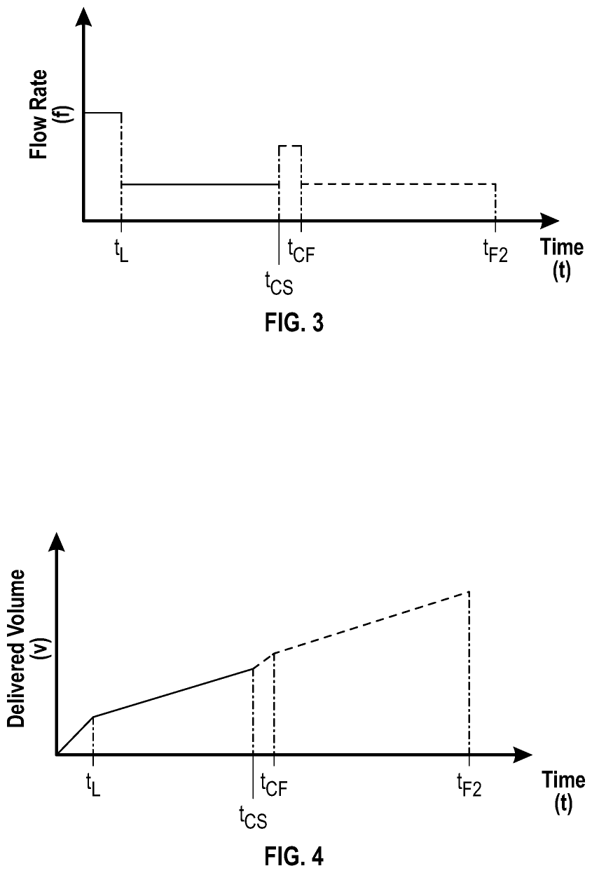

In certain cases, the modulation pattern can be modified while an infusion is running. For instance, in some cases it is possible for the user to program a bolus (which briefly increases the rate at which infusate is delivered to quickly increase the concentration of infusate in a patient's bloodstream) mid-infusion. Such a situation is illustrated in FIGS. 3 and 4. FIG. 3 illustrates the flow rate modulation pattern of FIG. 1, modified to account for a bolus requested at time t_{CS}. After the bolus concludes at time t_{CF}, the previously programmed basal rate resumes. It is noted that, here, the infusion is projected to end at an earlier time, t_{F2}, than time t_{F} in FIG. 1. FIG. 4 is a plot of infusate volume delivered to the patient as a function of time, corresponding to the flow rate modulation pattern of FIG. 3.

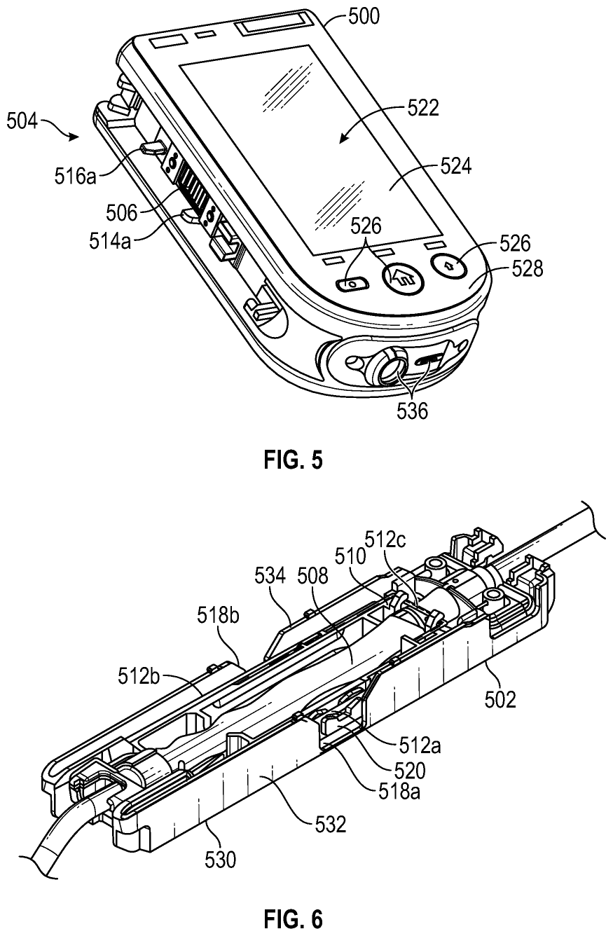

An example ambulatory infusion pump for delivering fluid in accordance with the modulation patterns of FIGS. 1 and 3 is described below with respect to FIGS. 5-11.

FIG. 5 depicts an example ambulatory peristaltic infusion pump 500, while FIG. 6 depicts an example cassette 502 for use with the ambulatory peristaltic infusion pump 500. The ambulatory peristaltic infusion pump 500 includes a receptacle 504 configured to receive the cassette 502. A peristaltic pump 506 within the receptacle 504 acts upon a tube 508 extending through a channel within the cassette 502 to pump fluid from a fluid container (e.g., a bag or a bottle; not shown) into a patient. An example free flow prevention clamp 510 is positioned within the cassette 502 to allow fluid flow through the tube 508 when the cassette 502 is coupled to the ambulatory peristaltic infusion pump 500 within the receptacle 504 (during which time the peristaltic pump 506 controls fluid flow through the tube 508) and to selectively cut off fluid flow through the tube 508 when the cassette 502 is not coupled to the ambulatory peristaltic infusion pump 500 in order to prevent unintentional fluid flow through the tube 508 (e.g., free flow).

The ambulatory peristaltic infusion pump 500 includes a user interface 522 for interacting with the ambulatory peristaltic infusion pump 500. The illustrated user interface 522 includes a display 524 (which may be a touchscreen) and buttons 526. A user controls operation of the ambulatory peristaltic infusion pump 500 via the user interface 522. The ambulatory peristaltic infusion pump 500 additionally includes a housing 528 for containing and supporting the components of the ambulatory peristaltic infusion pump 500 such as the peristaltic pump 506, electronics, power supplies, and the like.

The free flow prevention clamp 510 includes a first elongate section 512a, a second elongate section 512b, and a clamping section 512c. The housing 530 of the cassette 502 supports the free flow prevention clamp 510. The clamping section 512c is positioned within the cassette geometry such that, when the cassette 502 is received within the receptacle 504 of the ambulatory peristaltic infusion pump 500, the clamping section 512c extends across the channel receiving the tube 508. The housing 530 of the cassette 502 may be rigid plastic or other material capable of supporting the tube 508 and free flow prevention clamp 510.

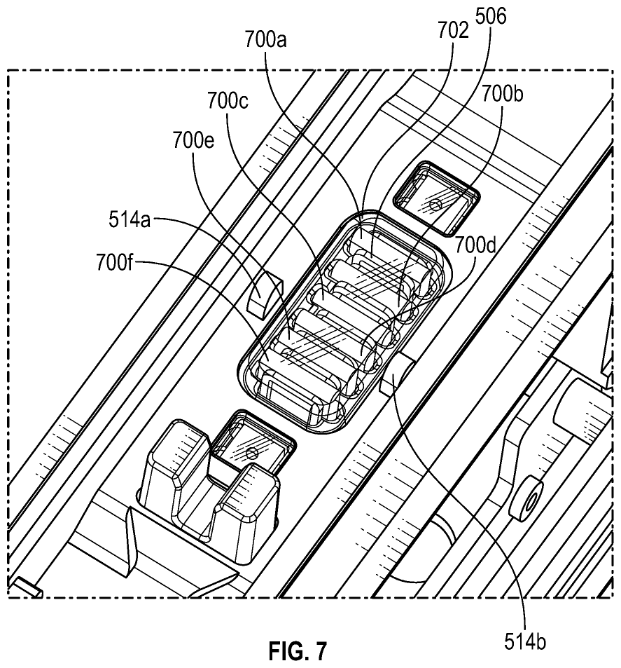

The ambulatory peristaltic infusion pump 500 also includes a pair of arc cams 514a and 514b (FIG. 7). First arc cam 514a is shown on one side of the receptacle illustrated in FIG. 5, but the second arc cam 514b is hidden from view. The pair of arc cams 514a and 514b engage the elongate sections 512a, 512b of the free flow prevention clamp 510 in order to lift the clamping section 512c. Additionally, the ambulatory pump 500 includes a pair of wedge cams 516a and 516b. A first wedge cam 516a is shown on one side of the receptacle 504 illustrated in FIG. 5, but the second wedge cam 516b is hidden from view. The pair of wedge cams 516a and 516b transition the free flow prevention clamp 510 from an open, manufactured/shipped state to an operational state, which is described in further detail below.

The cassette 502 also includes a first cutout 518a in a sidewall 532 of the cassette 502 and a second cutout 518b in an opposite sidewall 534 of the cassette 502. Additionally, the cassette 502 includes a touch pad 520 positioned on the first elongate section 512a adjacent a mid-point of the first elongate section 512a and the first cutout 518a. The touch pad 520 and cutout 518a together facilitate engagement of the first elongate section 512a by a finger of an operator in order to manually lift the clamping section 512c to allow fluid flow through the tube 508 (e.g., for priming the cassette 502) when the cassette 502 is not received within the receptacle 504 of the ambulatory peristaltic infusion pump 500. The touch pad 520 may be a press fit piece of rigid plastic. Although the touch pad 520 is illustrated as only on the first elongate section 512a, the touch pad 520 also may be provided on the second elongate section 512b.

The ambulatory infusion pump 500 further includes connector ports 536 that provide electronic access for control and for powering the ambulatory infusion pump 500 when used in the configurations described below.

FIG. 7 depicts the arc cams 514a and 514b and peristaltic pump 506 of the ambulatory peristaltic infusion pump 500. The peristaltic pump 506 includes multiple pump sliders 700 (six pump sliders 700a-f illustrated in FIG. 7). A flexible barrier (seal) 702 separates the pump sliders 700 (and other pump components of a pumping mechanism) from the receptacle 504 receiving the cassette 502 with the tube 508. The flexible barrier 702 provides a barrier between the fluid delivery apparatus/cassette and the pumping mechanism to prevent fluid from damaging components of the pumping mechanism.

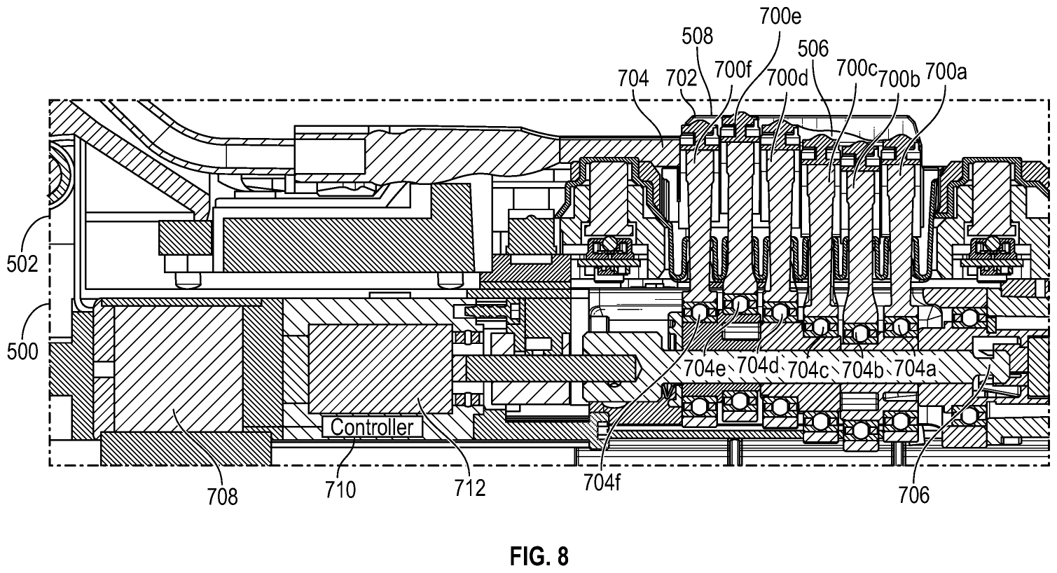

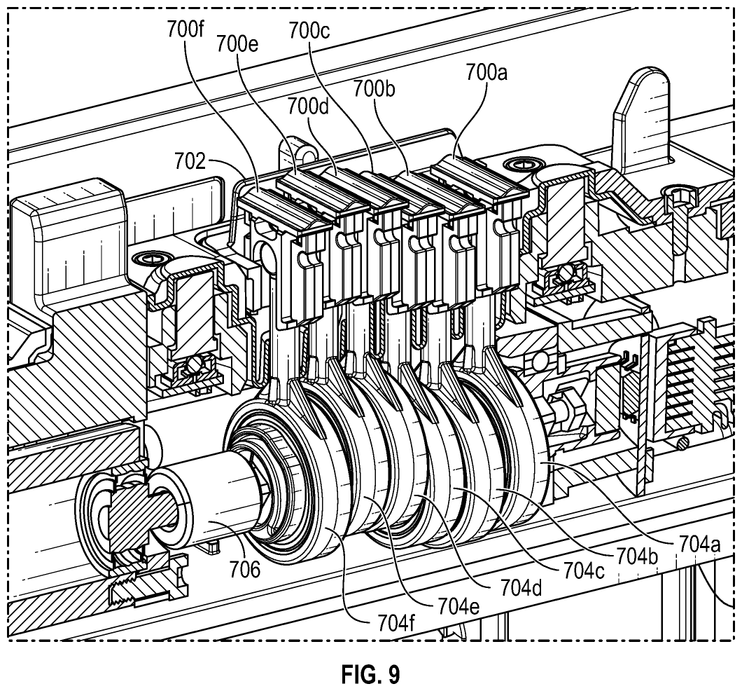

FIGS. 8 and 9 are cutaway views of the ambulatory peristaltic infusion pump 500 with the cassette 502 inserted into the receptacle 504 of the ambulatory infusion pump 500. Multiple cams 704 (six cams 704a-f) supported by a camshaft 706 act on respective pump sliders 700a-700f. The cams 704a-704f respectively raise and lower the pump sliders 700a-700f, which engage the tube 508 of the cassette 502 in order to force fluid though the tube 508. A pump motor 708 under control of a pump controller 710 turns the camshaft 706 by way of a gearbox 712. As the camshaft 706 turns, the cams 704a-700f, which are offset from each other in an axial direction, raise and lower respective pump sliders 700a-700f For example, cam 704a raises and lowers pump slider 700a; cam 704b raises and lowers pump slider 700b, and the like. The pump controller 710 may be a standalone or embedded processor configured to carry out instructions in order to control the operations of the ambulatory peristaltic infusion pump 500.

The pump controller 710 may include a user interface controller core/system controller core such as a dual core 32 bit processor from NXP of Eindhoven, Netherlands (e.g., model #MCIMX7S5EVM08SC), a pump controller core from NXP (e.g., model #MKV31F512VLH12), a supervisor controller core from NXP (e.g., model #MKL17Z128VMP4), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump camshaft revolutions per minute (RPM) corresponding to the infusion rate from a system controller core of the main processor. The microcontroller develops a pulse width modulation (PWM) motor drive parameter relating to the desired camshaft RPM. The PWM output of the microcontroller becomes the motor drive input to the pump motor driver, which contains motor drive transistors and protection circuitry. The rotation of the camshaft 706 of the pumping mechanism is measured by the magnetic encoder. At specified time intervals, the output of the encoder is read by the microcontroller, which uses the encoder value to compute the speed of the camshaft 706 and the position of the pump rotation. These values are then used to modify the PWM output to maintain the correct camshaft RPM.

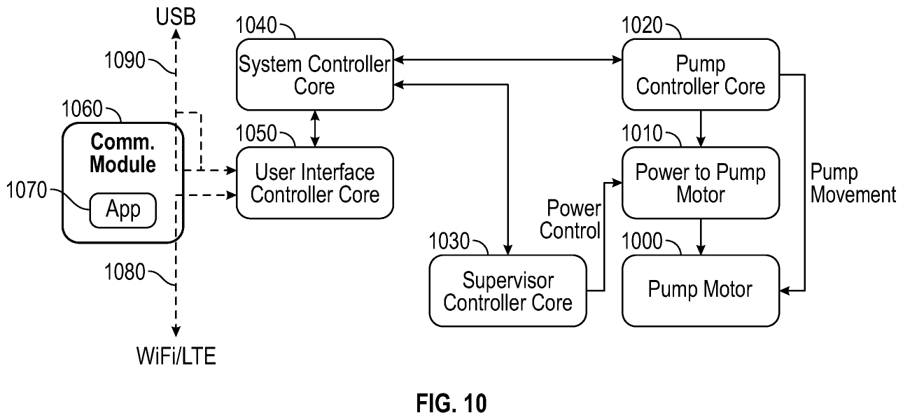

In sample configurations, the pump controller 710 includes multiple cores designed to ensure safe operation of the ambulatory peristaltic infusion pump 500. As described above with respect to FIGS. 5-9, the ambulatory peristaltic infusion pump 500 includes a peristaltic pump 506 configured to deliver fluid (e.g., pain medication) to a patient in response to pump controller 710. FIG. 10 is a diagram illustrating the communication module 1060 and multiple controller cores 1020-1050 used as pump controller 710 that together ensure safe operation of the peristaltic pump 506 in sample configurations. As shown in FIG. 10, the peristaltic pump 506 includes a pump motor 1000 that is powered by a power source 1010 and controlled by a collection of interconnected controller cores 1020-1050 that monitor operation of each other and process communications within the peristaltic pump 506.

In addition, multiple hardware and software external signaling safeguards are employed to ensure proper operation of the ambulatory peristaltic infusion pump 500 and to prevent the possibility of overinfusion (or underinfusion) due to unauthorized signals. For this purpose, all wireless communications with the ambulatory peristaltic infusion pump 500 pass through a communications module (“comm module”) 1060 that processes communications within the ambulatory peristaltic infusion pump 500. In addition to such hardware safeguards, software safeguards are implemented by a communications application (“comm app”) 1070 as well as pump side applications that validate the wired USB communication path to prevent unauthorized access to the ambulatory peristaltic infusion pump 500 that could interfere with proper operation.

In the configuration illustrated in FIG. 10, the pump controller core 1020 is dedicated to control movement of the pump motor 1000. The pump controller core 1020 may be located on a circuit board to which the pump motor 1000 is electrically connected. The pump core controller 1020 is responsible for controlling movement of the pump motor 1000 (e.g., start/stop/rotation rate/etc.). No other controller core has direct access to the pump motor 1000 (with the limited exception of the supervisor controller core 1030). The power to pump motor 1000 is provided by a pump motor driver (not shown). The supervisor controller core 1030 controls overall power to the pump motor driver, but the pump controller core 1020 also has the ability to enable/disable the pump motor driver, which in turn provides the power to the pump motor 1000.

The supervisor controller core 1030 is dedicated to supervisory operations for the pump motor 1000. The supervisor controller core 1030 can stop the pump motor 1000 by, for example, controlling the power to the pump motor at power source 1010 in the event any one of the other controller cores 1020, 1040, or 1050 is determined to be behaving improperly. The supervisor controller core 1030 monitors the health of all other controller cores in the ambulatory peristaltic infusion pump 500. In the event that a controller core is behaving improperly, the supervisor controller core 1030 is capable of stopping power flow to the pump motor 1000 by means of switching off the power source 1010 to the pump motor 1000. It will be appreciated that the pump motor 1000 may be stopped without cutting all power to the pump motor 1000.

The system controller core 1040 and user interface controller core 1050 control other operations of the ambulatory peristaltic infusion pump 500 (e.g., graphical user input/output, infusion state logic, alarms, configurations, etc.). Controller cores 1040 and 1050 are interconnected with each other. The system controller core 1040 is interconnected with the pump controller core 1020 and the supervisor controller core 1030. As noted above, if not explicitly notified of a problem, the supervisor controller core 1030 monitors the other controller cores for improper behavior and acts as appropriate.

The comm module 1060 includes the comm app 1070 that implements a wireless interface 1080 through which wireless communications must pass. The wired interface 1090 may connect directly to the user interface controller core 1050. The comm app 1070 checks certificates from external wireless signal sources that use the wireless communications interface 1080 to ensure the external signal source is authorized to communicate with the ambulatory peristaltic infusion pump 500 and that the certificates are valid (e.g., current and have not been revoked). Pump applications check the certificates on the wired interface path. Communications with the ambulatory peristaltic infusion pump 500 are only possible with a valid certificate. Messages from the wired interface 1090 also may be checked for valid certificates, but internal messages from known sources within the ambulatory peristaltic infusion pump 500 need not require such certificates provided physical access to the controller hardware is prevented.

Communications through the wired interface 1090 is only permitted when the user interface controller core 1050 indicates that the ambulatory peristaltic infusion pump 500 is not actively infusing. However, it is possible to connect the ambulatory peristaltic infusion pump 500 to an external source via the wireless interface 1090 to enable the ambulatory peristaltic infusion pump 500 to communicate with the external source to provide status information, to receive non-infusion-interrupting information for staging software and configuration updates, and to receive autoprogrammed infusions. On the other hand, when the ambulatory peristaltic infusion pump 500 is actively infusing, no communication is permitted through the wired interface 1090 to prevent a user from reconfiguring the ambulatory peristaltic infusion pump 500 during operation. It is noted that any autoprogramming information or staged software and configuration update information received via the wireless interface 1080 during an active infusion will not be applied during the current active infusion.

As a further signaling safeguard, all communications received by the ambulatory peristaltic infusion pump 500 from external signal sources are expected to utilize proprietary messaging protocols. If messages are received by the ambulatory peristaltic infusion pump 500 in any other format besides the proprietary messaging protocol, such messages are rejected, thus providing another level of signaling protection.

In addition, in sample configurations, the user interface controller core 1050 may have a different operating system than the supervisor controller core 1030, the pump controller core 1020, the system controller core 1040, and/or any other controller core. The use of different operating systems increases the complexity of the pump controller 710 so as to make it more difficult for malicious signals to pass due to the need to circumvent the safeguards of two or more operating systems.

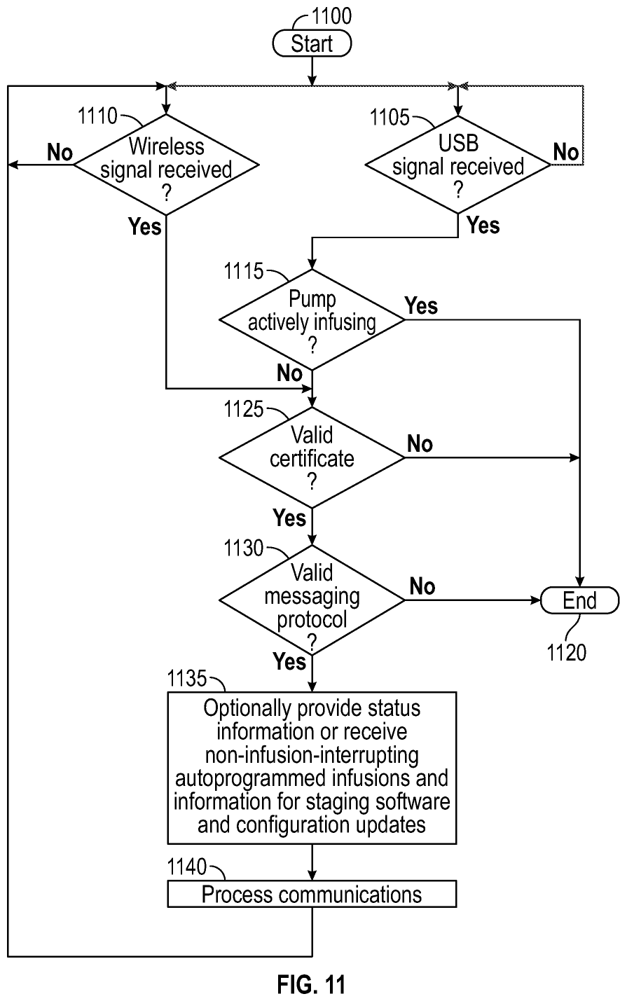

FIG. 11 is a flow chart illustrating the combined operation of the comm app 1070 of the comm module 1060 and software running on the user interface controller core 1050 for safe operation of the ambulatory peristaltic infusion pump 500 in a sample configuration.

As illustrated in FIG. 11, the comm app 1070 starts at 1100 and waits for receipt of an inbound communication signal via the wired interface 1090 at 1105 or the wireless interface 1080 at 1110. Upon receipt of an inbound wired communication signal, software running on the interface controller core 1050 checks at 1115 whether the ambulatory peristaltic infusion pump 500 is actively infusing. If so, the software running on the interface controller core 1050 will close any active communication links that may be open across the wired communication interface, and the process ends at 1120. If the ambulatory peristaltic infusion pump 500 is not actively infusing or if the communications are received via the wireless interface 1080, communications are permitted. Accordingly, the comm app 1070 checks at 1125 whether the received message came from a source with a valid certificate if the communications are over a wireless connection. Otherwise, if the message is received over a wired connection, then software running on the user interface controller core 1050 checks at 1125 if the received message came in from a source that has a valid certificate. If no valid certificate is received, the process ends at 1120. However, if a valid certificate has been received, the comm app 1070 checks at 1130 whether the received message has a valid messaging protocol. If not, the process ends at 1120. If the received message has a valid messaging protocol, communications are enabled. The comm app 1070 also may initiate outbound communications in order to report status information at 1135 to other elements via the wireless interface 1080. The comm app 1070 also may receive from external sources non-infusion-interrupting autoprogrammed infusions and information for staging software and configuration updates via the wireless interface 1080 at 1135.

Authorized communications are processed by the user interface controller core 1050 at 1140. The process repeats for each received message.

Though FIG. 11 illustrates that communications received from external sources via the wired interface 1090 are also checked for a valid certificate and a valid messaging protocol, it will be appreciated by those skilled in the art that these steps may be skipped for messages received via the wired interface 1090 from internal (i.e., known trustworthy) elements communicating with each other within the ambulatory peristaltic infusion pump 500. Also, limited internal pump communications may be permitted when the ambulatory peristaltic infusion pump 500 is actively infusing.

By separating the functionality of the pump controller 710 and providing signaling safeguards as described herein, operational control safeguards may be maintained whereby the pump motor 1000 is immediately stopped upon detection of improper behavior in any of the controller cores 1020-1050. Also, the hardware and software signaling safeguards described herein prevent unauthorized access to the pump controller 710 by potentially malicious signals from unauthorized parties or by viable signals interrupting ongoing infusions. These safeguards promote safe operation of the ambulatory peristaltic infusion pump 500 described herein as well as other types of infusion pumps known to those skilled in the art.

To prevent overinfusion, it is useful to program the ambulatory peristaltic infusion pump 500 to be able to restrict the volume of infusate that the patient can receive over a given period of time. To do this, in accordance with sample embodiments an analytical model of infusate volume delivered to the patient as a function of time can be utilized. It should be noted here that the sample embodiments are concerned specifically with those infusions for which flow rate varies discretely (rather than continuously), as it does in the examples shown in FIGS. 1 and 3. The analytical model may take the form of a piecewise function, v(t). During initial programming of an infusion, an initial version of the model is created. The model is updated each time the flow rate modulation pattern is modified over the course of the infusion. Each update to the model results in one or more new elements being added to the piecewise function.

To illustrate v(t), the sample flow rate modulation pattern described above with respect to FIG. 1 may be considered. The v(t) corresponding to this model (which is illustrated in FIG. 2) would be as shown in Equation 1. When the user modifies the flow rate modulation pattern as shown in FIG. 3, v(t) would change to Equation 2 (illustrated in FIG. 4).

$$v ( t ) = \{ \begin{matrix}{f}_{L} t , & 0 \leq t < {t}_{L} \\ {f}_{B} t + {V}_{1} , & {t}_{L} \leq t < {t}_{F}\end{matrix} \qquad Equation 1$$

$$v ( t ) = \{ \begin{matrix}{f}_{L} t , & 0 \leq t < {t}_{L} \\ {f}_{B} t + {V}_{1} , & {t}_{L} \leq t < {t}_{CS} \\ {f}_{C} t + {V}_{2} , & {t}_{CS} \leq t < {t}_{CF} \\ {f}_{B} t + {V}_{3} , & {t}_{CF} \leq t < {t}_{F 2}\end{matrix} \qquad Equation 2$$

Each time a version of the model is created, a function Δv(t), whose value for any given t is equal to the volume delivered over the past time Twill be generated. This function can be seen in Equation 3 below. This function, like the v(t) from which it is generated, will be piecewise. The maxima of each element of the piecewise Δv(t) is determined. The maxima is compared to a maximum permissible volume set by the user, and if any of the maxima meet or exceed the value, an alert will be issued to the user indicating as much (i.e., the user will be alerted that the patient is projected to receive more than the allowable volume of infusate). The user will be given the option to proceed anyway; however the volume infused over the past time Twill be monitored over the course of the infusion, and if the maximum permissible volume is reached, the infusion will be stopped.

$$Δ v ( t ) = v ( t ) - v ( t - T ) \qquad Equation 3$$

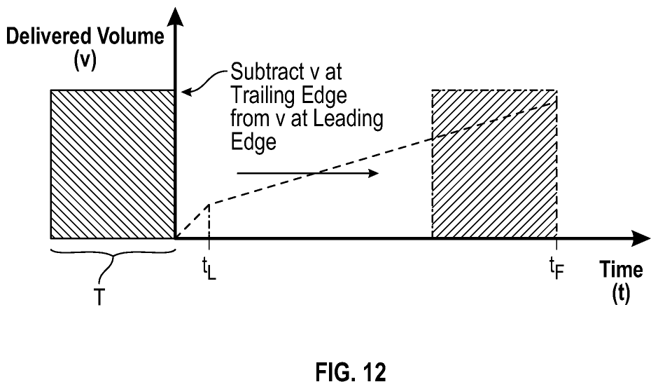

To illustrate Δv(t), the sample flow rate modulation pattern of FIG. 1 may again be considered. Generating a Δv(t) for this pattern is like sliding a window of length T along the corresponding plot of volume delivered (FIG. 2), and calculating the difference between the leading edge of the window and the trailing edge of the window. This is shown in FIG. 12, which is an illustration corresponding to Δv(t) for the flow rate modulation pattern of FIG. 1.

A memory-efficient means of evaluating Δv(t) would be to use a convolution integral, as shown in Equation 4 below. In Equation 4, u(t−τ) is a Boxcar function, with a width equal to T and a height equal to 1. The function ƒ(τ) is representative of the flow rate modulation pattern. If using Equation 3 to evaluate Δv(t), the y-intercept, slope, and domain are stored for each element of the piecewise v(t). However, if using Equation 4, only the slope and domain for each element are stored. This will result in less memory usage, which is helpful in cases where memory is limited and many modifications are made to the flow rate modulation pattern over the course of the infusion.

$$Δ v ( t ) = {\int }_{0}^{t} u ( t - τ ) f ( τ ) d τ \qquad Equation 4$$

By managing the infusion in accordance with Equation 3 or Equation 4, the flow rate modulation pattern may be modified multiple times without overfusion.

It should be noted that the method described works equally well if it is desired to restrict not the overall volume delivered to the patient, but rather the volume delivered by way of a subset of infusion components (for instance, it may be desirable to restrict the volume delivered by way of boluses alone). In this case, v(t) (and therefore Δv(t)) must account only for the infusion components that are of interest.

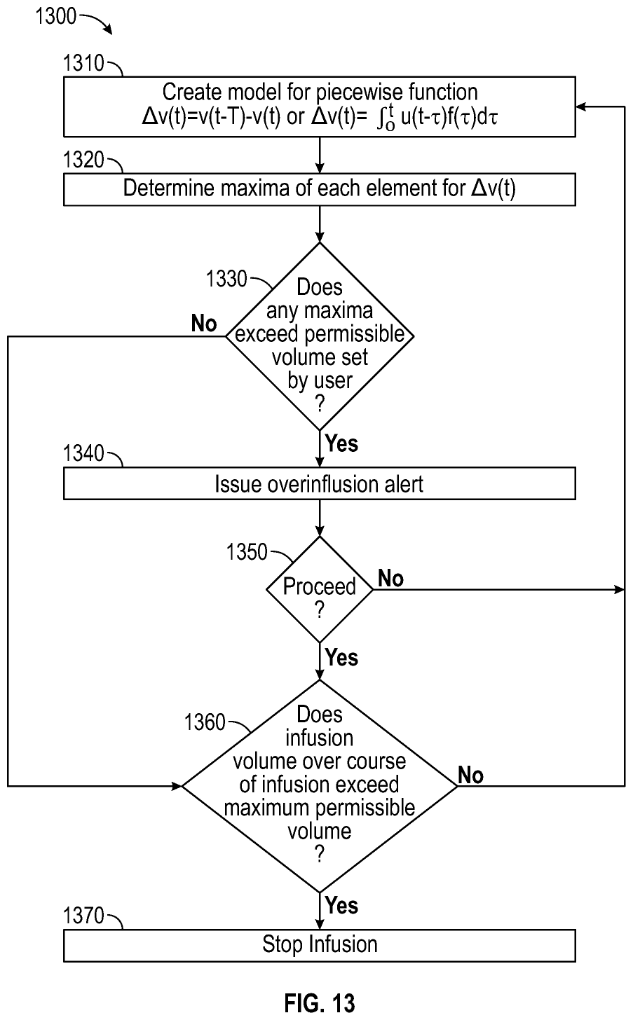

FIG. 13 is a flow chart illustrating a method 1300 for preventing overdosing by the infusion pump in a sample configuration.

As illustrated, the method starts at 1310 by creating a model in accordance with Equation 3 or Equation 4 whereby a piecewise function Δv(t) has a value for any given t that is equal to the volume delivered over the past time T The model accounts for any infusion components (such as loading doses and boluses) that are of interest. At 1320, the maxima of each element of the piecewise Δv(t)=v(t)−v(t−T) or Δv(t)=∫₀^{t}u(t−τ)ƒ(τ)dτ is determined. At 1330, the maxima from 1320 are compared to a maximum permissible volume set by the user, and if any of the maxima meet or exceed the maximum permissible volume, an alert will be issued to the user at 1340 indicating that the patient is projected to receive more than the allowable volume of infusate. The user will be given the option at 1350 to proceed. If the user wishes to proceed, the volume infused over the past time Twill be monitored over the course of the infusion at 1360, and if the maximum permissible volume is reached, the infusion will be stopped at 1370. Otherwise, the method returns to 1310 to update the model when prompted to do so (e.g., when further input is received from the user).

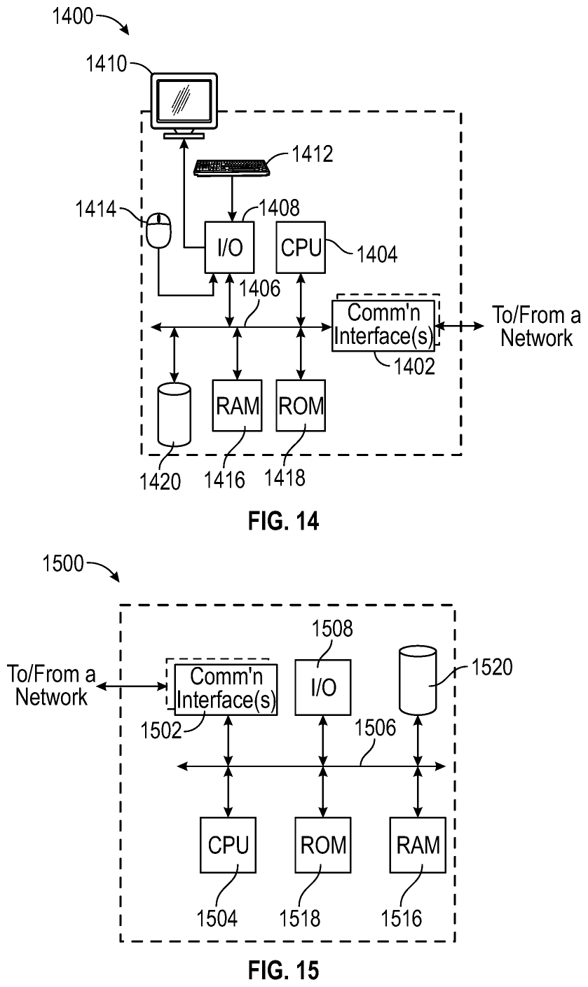

FIGS. 14 and 15 are functional block diagrams illustrating general-purpose computer hardware platforms configured to implement the functional examples described with respect to FIGS. 1-13 as discussed above.

Specifically, FIG. 14 illustrates an example computer platform 1400 and FIG. 15 depicts an example computer 1500 with user interface elements, as may be used to implement in a personal computer, ambulatory peristaltic infusion pump 100, or other type of workstation or terminal device. It is believed that those skilled in the art are familiar with the structure, programming and general operation of such computer equipment and as a result the drawings should be self-explanatory.

Hardware of an example computer platform 1400 (FIG. 14) includes a data communication interface 1402 for packet data communication. The computer platform 1400 also includes a central processing unit (CPU) 1404, in the form of circuitry forming one or more processors, for executing program instructions. The hardware of computer platform 1400 typically includes an internal communication bus 1406, program and/or data storage 1416, 1418, and 1420 for various programs and data files to be processed and/or communicated by the computer platform 1400, although the computer platform 1400 often receives programming and data via network communications. In one example, as shown in FIG. 14, the computer platform 1400 further includes a video display unit 1410 (e.g., a liquid crystal display (LCD) or a cathode ray tube (CRT)), an alphanumeric input device 1412 (e.g., a keyboard), a cursor control device 1414 (e.g., a mouse), each of which communicate with the internal communication bus 1406 via an input/output device (I/O) 1408. The hardware elements, operating systems and programming languages of such computer platforms 1400 are conventional in nature, and it is presumed that those skilled in the art are adequately familiar therewith. The computer platform 1400 may function as a server that may be implemented in a distributed fashion on a number of similar hardware platforms to distribute the processing load.

As illustrated in FIG. 15, hardware of a computer type user terminal device 1500, such as a PC (Personal Computer) or tablet computer, similarly includes a data communication interface 1502, CPU 1504, main memory 1516 and 1518, one or more mass storage devices 1520 for storing user data and the various executable programs, an internal communication bus 1506, and an input/output device (I/O) 1508.

Aspects of the methods for pump control, as outlined above, may be embodied in programming in general purpose computer hardware platforms (such as described above with respect to FIGS. 14 and 15), e.g. in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another, for example, from a processor 1404 of the system 1400 and/or from a pump controller 710 of a peristaltic infusion pump 500 to a computer or software of another system (not shown). Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables, copper wire and fiber optics, including the wires that include a bus within a computer system. Carrier-wave transmission media can take the form of electric or electromagnetic signals or acoustic or light waves such as those generated during radio frequency (RF) and light-based data communications. Common forms of computer-readable media therefore include, for example: a floppy disk, a flexible disk, hard disk, magnetic tape, any other magnetic medium, a CD-ROM (compact disc read only memory), DVD (Digital Video Disk) or DVD-ROM, any other optical medium, punch cards paper tape, any other physical storage medium with patterns of holes, a RAM (Random Access Memory), a PROM (Programmable Read Only Memory) and EPROM (Erasable Programmable Read Only Memory), a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render the computer system or device into a special-purpose machine that is customized to perform the operations specified in the program performed by the pump controller 710 of the pump 500.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 105 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. An infusion pump, comprising:
a pump motor configured to pump a liquid through a tube;
at least one pump controller for controlling the pump motor, the pump controller including a processor and a memory storing instructions therein that, when executed by the pump controller, perform operations including:
enabling input of a flow rate modulation pattern including a maximum permissible infusate volume;
creating a model including a piecewise function Δv(t) having a value for any given time t that is equal to a volume of fluid delivered by the pump motor to a user over a past time T, the model accounting for the flow rate modulation pattern including any infusion components specified in the flow rate modulation pattern;
determining a maxima of each element of the piecewise function Δv(t);
comparing the maxima of each element of the piecewise function Δv(t) to the maximum permissible volume;
when any of the maxima meet or exceed the maximum permissible volume, issuing an alert; and
controlling the pump motor in accordance with the flow rate modulation pattern to provide fluid to the user until the maximum permissible volume is reached.

2. The infusion pump of claim 1, wherein the processor of the at least one pump controller further executes instructions to perform additional operations including monitoring the volume of fluid delivered by the pump motor over the past time T over a course of an infusion, and when the maximum permissible volume is reached or the user chooses not to proceed, stopping the infusion.

3. The infusion pump of claim 1, wherein the processor of the at least one pump controller further executes instructions to perform additional operations including enabling the flow rate modulation pattern to be updated over the course of an infusion and updating the model each time the flow rate modulation pattern is updated by adding one or more new elements to the piecewise function Δv(t).

4. The infusion pump of claim 1, wherein the processor of the at least one pump controller executes the instructions to create the model including the piecewise function Δv(t)=v(t)−v(t−T).

5. The infusion pump of claim 1, wherein the processor of the at least one pump controller executes the instructions to create the model including the piecewise function Δv(t)=∫₀^{t}u(t−τ)ƒ(τ)dτ, where u(t−τ) is a Boxcar function with a width equal to T and a height equal to 1 and ƒ(τ) is a function representative of the flow rate modulation pattern.

6. A method of infusing a fluid into a user using an infusion pump, comprising:
enabling input of a flow rate modulation pattern including a maximum permissible infusate volume;
creating a model including a piecewise function Δv(t) having a value for any given time t that is equal to a volume of fluid delivered by the infusion pump to a user over a past time T, the model accounting for the flow rate modulation pattern including any infusion components specified in the flow rate modulation pattern;
determining a maxima of each element of the piecewise function Δv(t);
comparing the maxima of each element of the piecewise function Δv(t) to the maximum permissible volume;
when any of the maxima meet or exceed the maximum permissible volume, issuing an alert; and
controlling the infusion pump in accordance with the flow rate modulation pattern to provide fluid to the user until the maximum permissible volume is reached.

7. The method of claim 6, further comprising monitoring the volume of fluid delivered by the infusion pump over the past time T over a course of an infusion, and when the maximum permissible volume is reached or the user chooses not to proceed, stopping the infusion.

8. The method of claim 6, further comprising enabling the flow rate modulation pattern to be updated over the course of an infusion and updating the model each time the flow rate modulation pattern is updated by adding one or more new elements to the piecewise function Δv(t).

9. The method of claim 6, wherein the model includes the piecewise function Δv(t)=v(t)−v(t−T).

10. The method of claim 6, wherein the model includes the piecewise function Δv(t)=∫₀^{t}u(t−τ)ƒ(τ)dτ, where u(t−τ) is a Boxcar function with a width equal to T and a height equal to 1 and ƒ(τ) is a function representative of the flow rate modulation pattern.

11. A non-transitory controller-readable storage medium storing controller-executable instructions that, when executed by a processor of an infusion pump cause the infusion pump to perform operations comprising:
enabling input of a flow rate modulation pattern including a maximum permissible infusate volume;
creating a model including a piecewise function Δv(t) having a value for any given time t that is equal to a volume of fluid delivered by the infusion pump to a user over a past time T, the model accounting for the flow rate modulation pattern including any infusion components specified in the flow rate modulation pattern;
determining a maxima of each element of the piecewise function Δv(t);
comparing the maxima of each element of the piecewise function Δv(t) to the maximum permissible volume;
when any of the maxima meet or exceed the maximum permissible volume, issuing an alert; and
controlling the infusion pump in accordance with the flow rate modulation pattern to provide fluid to the user until the maximum permissible volume is reached.

12. The medium of claim 11, further comprising instructions that when executed by the processor of the infusion pump cause the infusion pump to perform additional operations comprising monitoring the volume of fluid delivered by the infusion pump over the past time T over a course of an infusion, and when the maximum permissible volume is reached or the user chooses not to proceed, stopping the infusion.

13. The medium of claim 11, further comprising instructions that when executed by the processor of the infusion pump cause the infusion pump to perform additional operations comprising enabling the flow rate modulation pattern to be updated over the course of an infusion and updating the model each time the flow rate modulation pattern is modified by adding one or more new elements to the piecewise function Δv(t).

14. The medium of claim 11, wherein the model includes the piecewise function Δv(t)=v(t)−v(t−T).

15. The medium of claim 11, wherein the model includes the piecewise function Δv(t)=∫₀^{t}u(t−τ)ƒ(τ)dτ, where u(t−τ) is a Boxcar function with a width equal to T and a height equal to 1 and ƒ(τ) is a function representative of the flow rate modulation pattern.
